SEQUENCE LISTING (16 sequences)

SEQ ID NO: 1 - DNA, 912 bp, RICKETTSIA PROWAZEKII
atgttaaaatcttcaaagcacgaagattctagtagtaaaaaaaatcaaaataataaattaatttttattgtacgacaattattctatctgataaaacatttttttagcaaaaccaaaacgcctgataatttttttggtattataaagcgtcttaaaattaatagccaaaaaatgtctttagacgagtttaatattttagctaatttattgaagctggaagataaaattgttgaagatatcatggtaccgcgttccgatattattgcaataaaattaactacgaatctagaagaattaagtgagtctattaagatagcagtcccacatacacgcacacttatatatgacggtactttggacaatgtagtaggattcattcatataaaggatttatttaaagcattggctacaaagcaaaatagtcctttaaaaagacttatacgtaaacatataattgcagctccttctatgaaattattggatttgttggcaaaaatgcgtcgtgagagaacacatattgcaatagttgtggatgagtatggtggtactgatggtttagttactattgaagatctaatagaagaaatagtagggcgaatagatgatgaacatgatcagcagttagatagcgctaattttaaagttattaataattcaacaattattgcgaatgcacgtattgaagtagaattgcttgaagaaataataggtgaaaagctaaaaaatgatgatgatgagtttgatacaataggtggactcgtcttgactagggtcagtagtgttccagctatcggtactagaatagatatttcagaaaatattgaaattgaagtaacagatgcaactcctcgttctttaaaacaagtcaaaattagattaaaaaacggtttgaatagtgataatttaacataa

SEQ ID NO: 2 - protein, 303 aa, RICKETTSIA PROWAZEKII
MLKSSKHEDSSSKKNQNNKLIFIVRQLFYLIKHFFSKTKTPDNFFGIIKRLKINSQKMSLDEFNILANLLKLEDKIVEDIMVPRSDIIAIKLTTNLEELSESIKIAVPHTRTLIYDGTLDNVVGFIHIKDLFKALATKQNSPLKRLIRKHIIAAPSMKLLDLLAKMRRERTHIAIVVDEYGGTDGLVTIEDLIEEIVGRIDDEHDQQLDSANFKVINNSTIIANARIEVELLEEIIGEKLKNDDDEFDTIGGLVLTRVSSVPAIGTRIDISENIEIEVTDATPRSLKQVKIRLKNGLNSDNLT

SEQ ID NO: 3 - DNA, 618 bp, RICKETTSIA PROWAZEKII
atgaagagcaaaaataataaatttatagcagtatctatctcctttattttgggaatcgctttaggtatttatgtagagagtacttattattttactaatataataaatagcaaatcattttctttatctaatgcacaaataaactattatagtatttcagaactttcaagaagtaatgtgagtacatgttttactccacctgccggttgtactaaatttatagtgcagcagatagagaaagctgaagaatctatttatatgcaggcatatggtatgagcgattcattaattactactgcacttattaatgcacagatgcgaggagtaaaagtgagaatattactcgatcgtagtaatttaaagcagaaattttctaaattatatgaattacaacaagcaaaaattgatgttggcatagatacggtacctggcattgcccataataaagttataattattgataaaaagaaagtaataactggttcgtttaatttcacggtttctgctgataaacgtaatgctgaaaatgtaattcttatagaagatcggaaattagcagagtcttatttacaaaattggttcagtcggaaaacaacgagtaatgcagtacatttttag

SEQ ID NO: 4 - protein, 205 aa, RICKETTSIA PROWAZEKII
MKSKNNKFIAVSISFILGIALGIYVESTYYFTNIINSKSFSLSNAQINYYSISELSRSNVSTCFTPPAGCTKFIVQQIEKAEESIYMQAYGMSDSLITTALINAQMRGVKVRILLDRSNLKQKFSKLYELQQAKIDVGIDTVPGIAHNKVIIIDKKKVITGSFNFTVSADKRNAENVILIEDRKLAESYLQNWFSRKTTSNAVHF

SEQ ID NO: 5 - DNA, 28 bp, ARTIFICIAL
  primer
tgggattaaggtgaaatagacatgatcc

SEQ ID NO: 6 - DNA, 27 bp, ARTIFICIAL
  primer
gccaattcttaattttatcacatacct

SEQ ID NO: 7 - DNA, 27 bp, ARTIFICIAL
  primer
cttattcttattttacttaatttgccg

SEQ ID NO: 8 - DNA, 24 bp, ARTIFICIAL
  primer
tattcctccaccacaacaaacagt

SEQ ID NO: 9 - DNA, 32 bp, ARTIFICIAL
  primer
attgaagctggaagataaaattgttgaagata

SEQ ID NO: 10 - DNA, 28 bp, ARTIFICIAL
  primer
tagcttttcacctattatttcttcaagc

SEQ ID NO: 11 - DNA, 25 bp, ARTIFICIAL
  primer
atgaagagcaaaaataataaattta

SEQ ID NO: 12 - DNA, 28 bp, ARTIFICIAL
  primer
ctaaaaatgtactgcattactcgttgtt

SEQ ID NO: 13 - DNA, 28 bp, ARTIFICIAL
  primer
acttttgagaatcattttattcatatgt

SEQ ID NO: 14 - DNA, 24 bp, ARTIFICIAL
  primer
ctatgttaaattatcactattcaa

SEQ ID NO: 15 - DNA, 24 bp, ARTIFICIAL
  primer
agtaatgagtggtttatgcaacga

SEQ ID NO: 16 - DNA, 28 bp, ARTIFICIAL
  primer
ctaaaaatgtactgcattactcgttgtt

(19) United States
(12) Patent Application Publication
(10) Pub. No.: US 2009/0297526 A1
(43) Pub. Date: Dec. 3, 2009

(54) Membranolytic Polypeptides and Methods of Use

(75) Inventors: Ted J. Whitworth, Galveston, TX (US); Donald H. Bouyer, League City, TX (US); David H. Walker, Galveston, TX (US)

Correspondence Address: MUETING, RAASCH & GEBHARDT, P.A., P.O. BOX 581336, MINNEAPOLIS, MN, 55458-1336, US

(73) Assignee: BOARD OF REGENTS, THE UNIVERSITY OF TEXAS SYSTEM, Austin, TX (US)

(21) Appl. No.: 11/921,695

(22) PCT Filed: Jun. 8, 2006
(86) PCT No.: PCT/US2006/022616

§ 371 Date: Aug. 4, 2009

Related U.S. Application Data
(60) Provisional application No. 60/688,520, filed on Jun. 8, 2005.

(51) Int. Cl. A61K 39/395; G01N 33/569; C07K 16/12; C12P 21/02; A61P 31/04

(52) U.S. Cl. 424/139.1; 436/501; 530/387.9; 800/6

(57) ABSTRACT

The present invention includes polypeptides, polynucleotides encoding the polypeptides, antibodies to the polypeptides, and methods for using the polypeptides and antibodies. The polypeptides include those with at least 80% identity to Rickettsia prowazekii TIyC or PId.

### CONTINUING APPLICATION DATA

This application claims the benefit of U.S. Provisional Application Ser. No. 60/688,520, filed Jun. 8, 2005, which is incorporated by reference herein.

### GOVERNMENT FUNDING

The present invention was made with government support under Grant Nos. U54 A1057156, RO1 AI21242, and T32 training grant 418151, each awarded by the National Institutes of Health. The Government may have certain rights in this invention.

### BACKGROUND

The genus Rickettsia are is gram-negative obligately intracellular bacteria, of which many cause zoonotic diseases in all continents inhabited by humans. Due to their strictly intracellular survival inside host cells, rickettsiae are classically transmitted to humans by arthropod vectors, which include ticks, mites, fleas, or lice. The pathogens causing human rickettsial diseases include the agent of epidemic typhus, Rickettsia prowazekii, which has resulted in the deaths of millions of people during wartime and natural disasters. The causative agents of spotted fever, e.g., R. rickettsii and R. conorii, are also included within this group.

R. prowazekii is the agent of epidemic typhus. It is transmitted by the human-body louse Pediculus humanus corporis and is also associated with flying squirrels and their ectoparasites (Bozeman et al., Am. J. Trop. Med. Hyg., 1981; 30:253-263; Duma et al., JAMA, 1981; 245:2318-2323). Infected lice, when feeding, excrete in their feces a live, dormant form of the organism onto the cutaneous bite sites. R. prowazekii enters the host through breaks in the epidermis caused by scratching of the contaminated area. Once within the host, R. prowazekii primarily infects endothelial cells and macrophages, where the organisms proliferate to massive numbers inside the host cells and eventually lyse them. The clinical symptoms of epidemic typhus include fever, severe headache, and myalgia. The nonspecific nature of the symptoms often leads to misdiagnosis, delaying appropriate chemotherapeutic intervention. The disease is fatal in approximately 30% of cases unless it is treated in a timely manner, preferably with a tetracycline drug (Azad et al., Ann. N.Y. Acad. Sci., 2003; 990:734-738). While the vegetative form of Rickettsia is generally unstable extracellularly, R. prowazekii in louse feces remains stable and infective for months or longer. Furthermore, there have been a multitude of well-documented laboratory-associated infections caused by accidental inhalation of aerosols containing R. prowazekii (Halle et al., J. Clin. Microbiol., 1980; 12:343-350). Owing to its high case fatality rate and the threat of human infection by low-dose, stable aerosol, R. prowazekii is on the select agent list that restricts possession and transfer of the agents, to hinder access to the organisms by would-be bioterrorists (Azad et al., Ann. N.Y. Acad. Sci., 2003; 990:734-738; Walker, Ann. N.Y. Acad. Sci., 2003; 990:739-742).

All members of the genus Rickettsia possess the ability to invade host cells and quickly escape phagosomal vacuoles before phagolysosomal fusion occurs and bactericidal mechanisms are activated (Feng et al., Infect. Immun., 2000; 68:6729-6736; Hackstadt, Infect. Agents Dis., 1996; 5:127-143; Teysseire et al., Infect. Immun., 1995; 63:366-374; Walker et al., Am. J. Trop. Med. Hyg., 2001; 65:936-942.). The mechanism of phagosomal escape remains unknown, although it has been hypothesized to be mediated by a hemolysin or phospholipase enzyme (Radulovic et al., Infect. Immun., 1999; 67:6104-6108; Renesto et al., J. Infect. Dis., 2003; 188:1276-1283). Genomic sequences for R. prowazekii, R. conorii, and R. typhi have revealed the presence of four genes with potential membranolytic activities: patatin B1 precursor, pat1 (RP602); hemolysin, tlyA (RP555); hemolysin C, tlyC (RP740); and pld (RP819) (Andersson et al., Nature, 1998; 396:133-140; McLeod et al., J. Bacteriol., 2004; 186:5842-5855; Ogata et al., Science, 2001; 293:2093-2098). The gene product of pld exhibits phospholipase D activity and has significant homology with the phospholipase D family of proteins (Renesto et al., J. Infect. Dis., 2003; 188:1276-1283). TlyC has been demonstrated to have hemolytic activity (Radulovic et al., Infect. Immun., 1999; 67:6104-6108). Rickettsiae have proven extremely difficult to manipulate genetically, hindering the production of site-directed knockout clones for the study of gene function (Baldridge et al., Appl. Environ. Microbiol., 2005; 71:2095-2105; Qin et al., Appl. Environ. Microbiol., 2004; 70:2816-2822; Rachek et al., J. Bacteriol., 1998; 180:2118-2124; Troyer et al., Infect. Immun., 1999; 67:3308-3311).

Prognosis in rickettsial infections depends in large part upon the initiation of antibiotic therapy early in the course of illness; however, symptoms of rickettsial diseases are similar to other infectious diseases, and there are no satisfactory diagnostic tests that permit the rapid and early diagnosis of a rickettsial infection.

### SUMMARY OF THE INVENTION

The present invention provides a method including providing a biological sample obtained from a subject suspected of being infected with an intracellular pathogen, such as a member of the genus Rickettsia, including an infection caused by R. prowazekii, contacting the biological sample with an antibody to form an antibody-polypeptide complex, wherein the antibody specifically binds a polypeptide having at least 80%, preferably, at least 95% identity to an amino acid sequence SEQ ID NO:2, and detecting the antibody-polypeptide complex, wherein the presence of the antibody-polypeptide complex indicates the presence of an intracellular pathogen, such as a member of the genus Rickettsia in the subject. The biological sample may be a blood sample, and the antibody may be a polyclonal antibody. In some aspects, the antibody does not specifically bind a polypeptide having less than 95% identity to the amino acid sequence SEQ ID NO:2.

The present invention also provides a method including providing a biological sample obtained from a subject suspected of being infected with an intracellular pathogen, such as a member of the genus Rickettsia, including an infection caused by R. prowazekii, contacting the biological sample with an antibody to form an antibody-polypeptide complex, wherein the antibody specifically binds a polypeptide having at least 80%, preferably, at least 95% identity to an amino acid sequence SEQ ID NO:4, and detecting the antibody-polypeptide complex, wherein the presence of the antibody-polypeptide complex indicates the presence of an intracellular pathogen, such as a member of the genus Rickettsia in the subject. The biological sample may be a blood sample, and the antibody may be a polyclonal antibody. In some aspects, the antibody does not specifically bind a polypeptide having less than 95% identity to the amino acid sequence SEQ ID NO:4.

Further provided is a method including administering an effective amount of a composition including an antibody to a subject, wherein the subject has or is at risk of having an infection by an intracellular pathogen, and wherein the antibody specifically binds a polypeptide having at least 80% identity, preferably, at least 95% identity, to an amino acid sequence SEQ ID NO:2, or having at least 80% identity, preferably, at least 95% identity, to an amino acid sequence SEQ ID NO:4. The intracellular pathogen may be a gram negative intracellular pathogen, such as a member of the genus Rickettsia.

The present invention provides a method including administering to a subject an effective amount of a composition containing a polypeptide, or a biologically active subunit or analog thereof, to a subject, wherein the amount administered is effective to result in an immune response. The polypeptide, or a biologically active subunit or analog thereof, has at least 80% identity, preferably, at least 95% identity, to an amino acid sequence SEQ ID NO:2 or SEQ ID NO:4.

The present invention further provides an antibody that specifically binds a polypeptide having at least 95% identity to an amino acid sequence SEQ ID NO:2 and does not specifically bind a polypeptide having less than 95% identity to the amino acid sequence SEQ ID NO:2, and an antibody that specifically binds a polypeptide having at least 95% identity to an amino acid sequence SEQ ID NO:4 and does not specifically bind a polypeptide having less than 95% identity to the amino acid sequence SEQ ID NO:4. Also included are compositions that include the antibody, methods for making such antibodies, and kits that include such antibodies.

The terms “comprises” and variations thereof do not have a limiting meaning where these terms appear in the description and claims. Unless otherwise specified, “a,” “an,” “the,” and “at least one” are used interchangeably and mean one or more than one.

### BRIEF DESCRIPTION OF THE FIGURES

FIG. 1. Proportions of R. prowazekii organisms within Vero cell cytosol at 30 and 50 minutes postinfection. The difference between proportions at 30 minutes and 50 minutes postinfection was statistically significant (P<0.001).

FIG. 2. mRNA expression of four potentially membranolytic genes of Rickettsia prowazekii in Vero cell culture at 30 minutes postinfection. (A) RT-PCR using primers specific for tlyA, pat1, tlyC, and pld. (B) PCRs using R. prowazekii DNA demonstrated the effectiveness of the primers. Lane 1, negative control (tlyA primers with water as the template); lane 2, primers specific for tlyA; lane 3, primers specific for pat1; lane 4, primers specific for tlyC; lane 5, primers specific for pld.

FIG. 3. mRNA clonal expression of rickettsial genes in Salmonella after overnight incubation. (A) RT-PCR using primers specific for tlyC. (B) RT-PCR using primers specific for pld. Lane 1, negative control (RT-PCR using RNA extracted from SB109); lane 2, recombinant Salmonella RNA expressing tlyC (A) or pld (B); lane 3, RT-PCR of RNA with no reverse transcriptase enzyme.

FIG. 4. Electron photomicrographs of Salmonella organisms and transformants. (A) Two wild-type salmonellae 4 hours postinfection. One is entering the cell (arrowhead), and the other is completely surrounded by a host cell vacuolar membrane (arrow). Bar, 0.5 μm. (B) Salmonella transformed with plasmid only located completely within a host cell vacuole. Bar, 0.5 μm. (C) Salmonella tlyC transformants at the site of the remnants of a host cell vacuole. Vacuolar membrane breaks are indicated by arrowheads. Bar, 0.5 μm. (D) Salmonella pld transformants are all located free within the cytosol of the host cell. Two organisms are identified with arrows. Bar, 1.0 μm. (E) Higher magnification of two Salmonella tlyC transformants (arrows) present within the host cell cytosol. Bar, 0.5 μm. (F) Higher magnification of Salmonella pld transformants within the host cell cytosol. Bar, 0.5 μm.

FIG. 5. Quantification of vacuolar escape by the Salmonella transformants. SB109, nontransformed Salmonella; tlyC, Salmonella tlyC transformants; pld, Salmonella pld transformants; NI (not infected), Salmonella containing plasmid only. The percentage of intracytosolic salmonellae transcriptionally expressing tlyC or pld was significantly different from that of the nontransformed salmonellae.

FIG. 6. Nucleotide sequence of a tlyC coding region (SEQ ID NO:1) and the TlyC protein (SEQ ID NO:2) it encodes.

FIG. 7. Nucleotide sequence of a pld (SEQ ID NO:3) coding region and the Pld protein (SEQ ID NO:4) it encodes.

### DETAILED DESCRIPTION OF ILLUSTRATIVE EMBODIMENTS

The present invention provides polypeptides, antibodies to the polypeptides, and methods for using the polypeptides and antibodies. As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

If naturally occurring, a polypeptide is preferably isolated, more preferably, purified. An “isolated” compound, such as a polypeptide, is one that is separate and discrete from its natural environment. A “purified” compound, such as a polypeptide, is one that is at least 60% free, preferably 75% free, and most preferably 90% free from other components with which they are naturally associated. Compounds such as polypeptides and polynucleotides that are produced outside the organism in which they naturally occur, e.g., through chemical or recombinant means, are considered to be isolated and purified by definition, since they were never present in a natural environment.

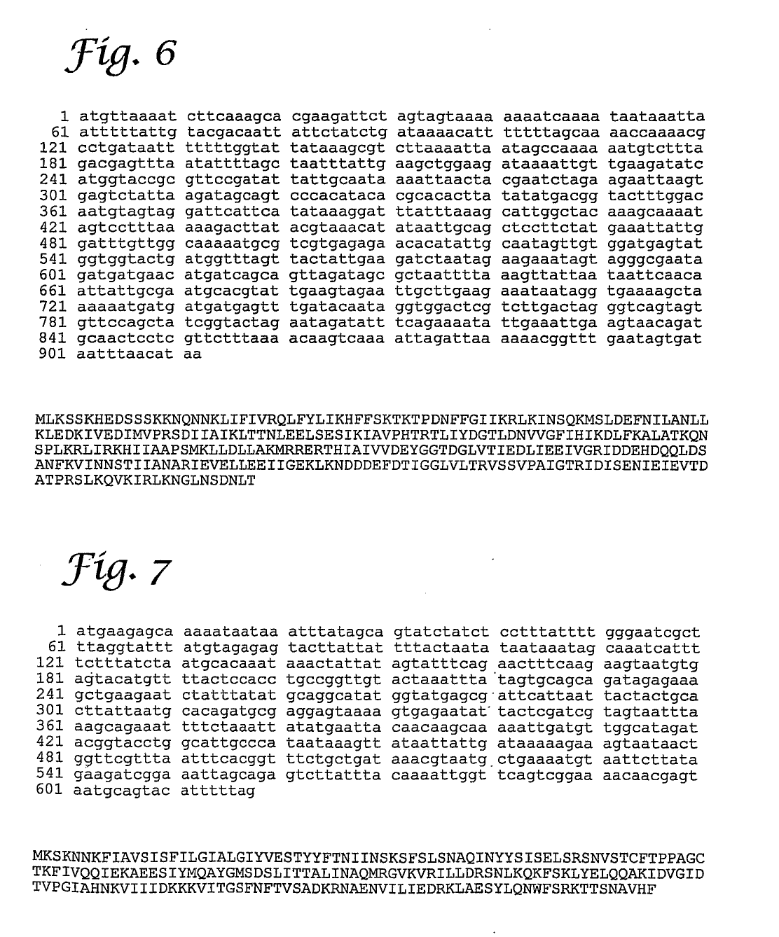

In one aspect, the polypeptide is a polypeptide having the amino acid sequence depicted at SEQ ID NO:2 (FIG. 6, Genbank accession number CAA15168), which can be referred to as a TlyC polypeptide. In another aspect, the polypeptide is a polypeptide having the amino acid sequence depicted at SEQ ID NO:4 (FIG. 7, Genbank accession number CAA15244), which can be referred to as a Pld polypeptide. Also included are polypeptides having similarity with the amino acid sequence of SEQ ID NO:2 or SEQ ID NO:4. The similarity is referred to as structural similarity and is generally determined by aligning the residues of the two amino acid sequences (i.e., a candidate amino acid sequence and the amino acid sequence of SEQ ID NO:2 or SEQ ID NO:4) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence is the amino acid sequence being compared to an amino acid sequence present in SEQ ID NO:2 or SEQ ID NO:4. A candidate amino acid sequence can be isolated from a microbe, such as a member of the genus Rickettsia, for instance R. prowazekii, or can be produced using recombinant techniques, or chemically or enzymatically synthesized. Preferably, two amino acid sequences are compared using the BESTFIT algorithm in the GCG package (version 10.2, Madison Wis.), or, more preferably, the Blastp program of the BLAST 2 search algorithm, as described by Tatusova, et al. (FEMS Microbiol Lett 1999, 174:247-250), and available through the World Wide Web, for instance at the internet site maintained by the National Center for Biotechnology Information, National Institutes of Health. Preferably, the default values for all BLAST 2 search parameters are used, including matrix=BLOSUM62; open gap penalty=11, extension gap penalty=1, gap x_dropoff=50, expect=10, wordsize=3, and optionally, filter on. In the comparison of two amino acid sequences using the BLAST search algorithm, structural similarity is referred to as “identities.” Preferably, a TlyC polypeptide or a Pld polypeptide useful in the present invention includes polypeptides with an amino acid sequence having at least 80% amino acid identity, at least 85% amino acid identity, at least 90% amino acid identity, or at least 95% amino acid identity to SEQ ID NO:2 or SEQ ID NO:4.

A polypeptide useful in the present invention may be biologically active. A “biologically active” polypeptide is a polypeptide that exhibits membranolytic activity. Methods for determining whether a polypeptide exhibits membranolytic activity are known in the art and used routinely. An example of a method for determining the activity of a candidate TlyC polypeptide uses the lysis of erythrocytes, such as sheep erythrocytes. The lysis of erythrocytes is also referred to a hemolytic activity. The method includes serially diluting a sample containing the candidate polypeptide in a suitable diluent, for instance, 10 mM Tris-HCL (pH 7.4), 0.9% NaCl. A portion of each sample is then added to an equal volume of 2% erythrocytes, and incubated at 20° C. to 27° C. for 1 to 2 hours. The titer of hemolytic activity can be expressed as the highest dilution causing complete lysis of the erythrocytes. The sample of candidate polypeptide can be obtained by, for instance, lysis of a cell expressing the candidate polypeptide, or, in some cases, possibly by using a supernatant from such a cell grown in culture. An example of a method for determining the activity of a candidate Pld polypeptide uses the generation of water soluble radioactivity from [³H]-phosphatidylcholine (PC). The method includes creating liposomes with a mixture of [³H]-PC (1,3 phosphatidyl[N-methyl-3H]choline, 1,2 dipalmitoyl) and unlabeled phosphatidylcholine (PC), for instance, by sonication. The ratio can vary, and in one example, the ratio can be 1 mmol/L PC: 10⁶ cpm/mL [³H]-PC, where the [³H]-PC is 86 Ci/mmol. A sample of candidate polypeptide is incubated in an appropriate buffer, for instance, 50 mmol/L Tris, 80 mmol/L KCl, and 5 mmol/L CaCl₂ (pH 8), with the liposomes. After stopping the reaction, for instance, by the addition of a mixture of chloroform/methanol/HCl, the amount of radioactivity in the aqueous phase can be analyzed by standard methods, for instance by thin-layer chromatography. The sample of candidate polypeptide can be obtained by, for instance, lysis of a cell expressing the candidate polypeptide, or, in some cases, possibly by using a supernatant from such a cell grown in culture. Other methods for determining whether a polypeptide exhibits membranolytic activity include expressing a candidate polypeptide in Salmonella and evaluating the ability of the Salmonella to escape phagosomal vacuoles (see Example 1).

Preferably, a polypeptide of the present invention has immunogenic activity. “Immunogenic activity” refers to an amino acid sequence which elicits an immunological response in a subject. An immunological response to a polypeptide is the development in a subject of a cellular and/or antibody-mediated immune response to the polypeptide. Usually, an immunological response includes but is not limited to one or more of the following effects: the production of antibodies, B cells, helper T cells, suppressor T cells, and/or cytotoxic T cells, directed specifically to an epitope or epitopes of the polypeptide. The immunogenic activity may be protective. “Protective immunogenic activity” refers to the ability of a polypeptide to elicit an immunological response in an animal that prevents or inhibits infection by a member of the genus Rickettsia, for instance, R. prowazekii. Whether a polypeptide has protective immunogenic activity can be determined by methods known in the art (see, for instance, Walker et al., Lab. Invest., 2000; 80:1361-72; Diaz-Montero et al., Am. J. Trop. Med. Hyg., 2001; 65:371-78; and Crocquet-Valdes et al., Vaccine, 2002; 20:979-88).

Polypeptides useful in the present invention also include biologically active subunits and analogs of TlyC polypeptides and Pld polypeptides. A biologically active “subunit” of a TlyC polypeptide or Pld polypeptide includes a TlyC polypeptide or a Pld polypeptide that has been truncated at either the N-terminus, or the C-terminus, or both, by one or more amino acids, as long as the truncated polypeptide retains biological activity, and, in the case of a TlyC subunit, contains at least 150 amino acids, at least 200 amino acids, or at least 250 amino acids, or in the case of a Pld subunit, contains at least 50 amino acids, at least 100 amino acids, or at least 150 amino acids

A biologically active “analog” of a polypeptide of the invention includes a TlyC polypeptide or a Pld polypeptide that has been modified by the addition, substitution, or deletion of one or more contiguous or noncontiguous amino acids, or that has been chemically or enzymatically modified, e.g., by attachment of a reporter group, by an N-terminal, C-terminal or other functional group modification or derivatization, or by cyclization, as long as the analog retains biological activity. An analog can thus include additional amino acids at one or both of the termini of a polypeptide.

A TlyC polypeptide or a Pld polypeptide having a substitution is a polypeptide having an amino acid residue in the polypeptide that is removed and a different residue inserted in its place. Substitutes for an amino acid in a TlyC polypeptide or a Pld polypeptide are preferably conservative substitutions. The sites of greatest interest for conservative substitutions include sites identified as the antigenic determining region(s), and the active site(s). Other sites of interest are those in which particular residues obtained from various species are identical. These positions may be important for biological activity. A conservative substitution is typically the substitution of one amino acid for another that is a member of the same class. For example, it is well-known in the art of protein biochemistry that an amino acid belonging to a grouping of amino acids having a particular size or characteristic (such as charge, hydrophobicity, and/or hydrophilicity) can generally be substituted for another amino acid without substantially altering the structure of a polypeptide. For the purposes of this invention, conservative amino acid substitutions are defined to result from exchange of amino acids residues from within one of the following classes of residues: Class I: Ala, Gly, Ser, Thr, and Pro (representing small aliphatic side chains and hydroxyl group side chains); Class II: Cys, Ser, Thr and Tyr (representing side chains including an —OH or —SH group); Class III: Glu, Asp, Asn and Gln (carboxyl or amino group containing side chains); Class IV: His, Arg and Lys (representing basic side chains); Class V: Ile, Val, Leu, Phe and Met (representing hydrophobic side chains); and Class VI: Phe, Trp, Tyr and His (representing aromatic side chains). The classes also include related amino acids such as 3-hydroxyproline and 4-hydroxyproline in Class I; homocysteine in Class II; 2-aminoadipic acid, 2-aminopimelic acid, γ-carboxyglutamic acid, β-carboxyaspartic acid, and the corresponding amino acid amides in Class III; ornithine, homoarginine, N-methyl lysine, dimethyl lysine, trimethyl lysine, 2,3-diaminopropionic acid, 2,4-diaminobutyric acid, homoarginine, sarcosine and hydroxylysine in Class IV; substituted phenylalanines, norleucine, norvaline, 2-aminooctanoic acid, 2-aminoheptanoic acid, statine and β-valine in Class V; and naphthylalanines, substituted phenylalanines, tetrahydroisoquinoline-3-carboxylic acid, and halogenated tyrosines in Class VI.

Polynucleotides encoding the polypeptides described herein, as well as subunits and analogs thereof, are also included in the present invention. As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, including for instance coding sequences, and non-coding sequences such as regulatory sequences. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. A polynucleotide may be isolated, preferably, purified. A coding sequence, also referred to herein as a coding region, is a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end

Preferred polynucleotides include polynucleotides encoding the polypeptide depicted at SEQ ID NO:2 or SEQ ID NO:4, and the complement of such polynucleotide, as well as polynucleotides having a nucleotide sequence that is “substantially complementary” to (a) a nucleotide sequence that encodes polypeptide sequences SEQ ID NO:2 or SEQ ID NO:4, or (b) the complement of such nucleotide sequence. “Substantially complementary” polynucleotides can include at least one base pair mismatch, however the two polynucleotides will still have the capacity to hybridize. For instance, the middle nucleotide of each of the two DNA molecules 5′-AGCAAATAT and 5′-ATATATGCT will not base pair, but these two polynucleotides are nonetheless substantially complementary as defined herein. Two polynucleotides are substantially complementary if they hybridize under hybridization conditions exemplified by 2×SSC (SSC: 150 mM NaCl, 15 mM trisodium citrate, pH 7.6) at 55° C. Substantially complementary polynucleotides for purposes of the present invention preferably share at least one region of at least 20 nucleotides in length, more preferably, at least 100 nucleotides in length, which shared region has at least 60% nucleotide identity, at least 80% nucleotide identity, at least 90% nucleotide identity, or at least 95% nucleotide identity. Particularly preferred substantially complementary polynucleotides share a plurality of such regions.

Other preferred polynucleotides include those having similarity with the nucleotide sequence of SEQ ID NO: 1 (Genbank accession number AJ235273, region: complement of 54966-55877) or SEQ ID NO:3 (Genbank accession number AJ235273, region: complement of 153538-154155). The similarity is referred to as structural similarity and is determined by aligning the residues of the two polynucleotides (i.e., the nucleotide sequence of the candidate coding region and the nucleotide sequence of the coding region of SEQ ID NO: 1 or SEQ ID NO:3) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate coding region is the coding region being compared to a coding region present in SEQ ID NO: 1 or SEQ ID NO:3. A candidate nucleotide sequence can be isolated from a microbe, such as a member of the genus Rickettsia, for instance R. prowazekii, or can be produced using recombinant techniques, or chemically or enzymatically synthesized. Preferably, two nucleotide sequences are compared using the BESTFIT algorithm in the GCG package (version 10.2, Madison Wis.), or, more preferably, the Blastn program of the BLAST 2 search algorithm, as described by Tatusova, et al. (FEMS Microbiol Lett., 1999; 174:247-250), and available through the World Wide Web, for instance at the internet site maintained by the National Center for Biotechnology Information, National Institutes of Health. Preferably, the default values for all BLAST 2 search parameters are used, including reward for match=1, penalty for mismatch=−2, open gap penalty=5, extension gap penalty=2, gap x_dropoff=50, expect=10, wordsize=11, and optionally, filter oil. In the comparison of two nucleotide sequences using the BLAST search algorithm, structural similarity is referred to as “identities.” Preferably, a polynucleotide includes a nucleotide sequence having at least 80% nucleotide identity, at least 85% nucleotide identity, at least 90% nucleotide identity, or at least 95% nucleotide identity to the coding region of SEQ ID NO: 1 or SEQ ID NO:3.

It should be understood that a polynucleotide encoding a polypeptide represented by SEQ ID NO:2 or SEQ ID NO:4 is not limited to the nucleotide sequence disclosed at SEQ ID NO:1 or SEQ ID NO:3, but also includes the class of polynucleotides encoding such polypeptides as a result of the degeneracy of the genetic code. For example, the naturally occurring nucleotide sequence SEQ ID NO:1 is but one member of the class of nucleotide sequences encoding a polypeptide having amino acid SEQ ID NO:2. Likewise, SEQ ID NO:3 is but one member of the class of nucleotide sequences encoding a polypeptide having amino acid SEQ ID NO:4. The class of nucleotide sequences encoding a selected polypeptide sequence is large but finite, and the nucleotide sequence of each member of the class can be readily determined by one skilled in the art by reference to the standard genetic code, wherein different nucleotide triplets (codons) are known to encode the same amino acid. Likewise, a polynucleotide of the invention encoding a biologically active subunit or analog of the TlyC polypeptide or the Pld polypeptide includes the multiple members of the class of polynucleotides encoding the selected polypeptide sequence.

Moreover, a polynucleotide that “encodes” a polypeptide described herein optionally includes both coding and noncoding regions, and it should therefore be understood that, unless expressly stated to the contrary, a polynucleotide that “encodes” a polypeptide is not structurally limited to nucleotide sequences that encode a polypeptide but can include other nucleotide sequences outside (i.e., 5′ or 3′ to) the coding region.

A polynucleotide encoding a polypeptide described herein can be inserted in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual, Cold Spring Harbor Laboratory Press, 1989. A vector can provide for further cloning (amplification of the polynucleotide), i.e., a cloning vector, or for expression of the polypeptide encoded by the coding region, i.e., an expression vector. The term vector includes, but is not limited to, plasmid vectors, viral vectors, cosmid vectors, or artificial chromosome vectors. Typically, a vector is capable of replication in a bacterial host, for instance E. coli or S. typhimurium. Preferably the vector is a plasmid.

Selection of a vector depends upon a variety of desired characteristics in the resulting construct, such as a selection marker, vector replication rate, and the like. Suitable host cells for cloning or expressing the vectors herein are prokaryote or eukaryotic cells. Preferably the host cell secretes minimal amounts of proteolytic enzymes. Suitable prokaryotes include eubacteria, such as gram-negative organisms, for example, E. coli or S. typhimurium.

An expression vector optionally includes regulatory sequences operably linked to the coding region. The invention is not limited by the use of any particular promoter, and a wide variety of promoters are known. Promoters act as regulatory signals that bind RNA polymerase in a cell to initiate transcription of a downstream (3′ direction) coding region. The promoter used can be a constitutive or an inducible promoter. It can be, but need not be, heterologous with respect to the host cell. In one aspect, the promoter is a promoter normally present in a R. prowasekii, upstream of the coding sequences encoding a TlyC polypeptide or a Pld polypeptide.

An expression vector can optionally include a ribosome binding site and a start site (e.g., the codon ATG) to initiate translation of the transcribed message to produce the polypeptide. It can also include a termination sequence to end translation. A termination sequence is typically a codon for which there exists no corresponding aminoacetyl-tRNA, thus ending polypeptide synthesis. The polynucleotide used to transform the host cell can optionally further include a transcription termination sequence.

The polynucleotide used to transform a host cell optionally includes one or more marker sequences, which typically encode a molecule that inactivates or otherwise detects or is detected by a compound in the growth medium. For example, the inclusion of a marker sequence can render the transformed cell resistant to an antibiotic, or it can confer compound-specific metabolism on the transformed cell. Examples of a marker sequence are sequences that confer resistance to kanamycin, ampicillin, chloramphenicol, tetracycline, and neomycin.

Polypeptides, and biologically active subunits and analogs thereof, useful in the present invention can be produced using recombinant DNA techniques, such as an expression vector present in a cell. Such methods are routine and known in the art. The polypeptides, and subunits and analogs thereof, can also be synthesized in vitro, e.g., by solid phase peptide synthetic methods. The solid phase peptide synthetic methods are routine and known in the art. A polypeptide produced using recombinant techniques or by solid phase peptide synthetic methods can be further purified by routine methods, such as fractionation on immunoaffinity or ion-exchange columns, ethanol precipitation, reverse phase HPLC, chromatography on silica or on an anion-exchange resin such as DEAE, chromatofocusing, SDS-PAGE, ammonium sulfate precipitation, gel filtration using, for example, Sephadex G-75, or ligand affinity chromatography.

The present invention also provides antibodies, including monoclonal and polyclonal antibodies, that specifically bind a TlyC polypeptide or a Pld polypeptide described herein, or a biologically active subunit or analog thereof. The term “antibody,” unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanized antibodies.

As used herein, an antibody that can “specifically bind” a polypeptide is an antibody that interacts only with the epitope of the antigen that induced the synthesis of the antibody, or interacts with a structurally related epitope. An antibody that “specifically binds” to an epitope will, under the appropriate conditions, interact with the epitope even in the presence of a diversity of potential binding targets. As used herein, the term “polypeptide:antibody complex” refers to the complex that results when an antibody specifically binds to a polypeptide, or a subunit or analog thereof. In some aspects, antibodies useful in the present invention include those that specifically bind to a polypeptide having at least 80%, at least 85%, at least 90%, at least 95%, at least 96%, at least 97%, at least 98%, at least 99%, or 100% identity with the amino acid sequence depicted at SEQ ID NO:2, SEQ ID NO:4, or a subunit or analog thereof. Optionally, in some aspects, antibodies useful in the present invention do not specifically bind to a polypeptide having less than 99%, less than 98%, less than 97%, less than 96%, less than 95%, less than 94%, less than 93%, or less than 92% identity with the amino acid sequence depicted at SEQ ID NO:2, SEQ ID NO:4, or a subunit or analog thereof.

Antibodies of the present invention can be prepared using the intact polypeptide or biologically active subunits or analogs thereof as the immunizing agent. Optionally, a polypeptide described herein, or a subunit or analog thereof, can be covalently bound or conjugated to a carrier polypeptide to improve the immunological properties of the polypeptide. Useful carrier polypeptides are known in the art.

The preparation of polyclonal antibodies is well known. Polyclonal antibodies may be obtained by immunizing a variety of warm-blooded animals such as horses, cows, goats, sheep, dogs, chickens, rabbits, mice, hamsters, guinea pigs and rats as well as transgenic animals such as transgenic sheep, cows, goats or pigs, with an immunogen. The resulting antibodies may be isolated from other proteins by using an affinity column having an Fc binding moiety, such as protein A, or the like.

Monoclonal antibodies can be obtained by various techniques familiar to those skilled in the art. Briefly, spleen cells from an animal immunized with a desired antigen are immortalized, commonly by fusion with a myeloma cell (see, for example, Antibodies: A Laboratory Manual, Harlow et al., Eds., Cold Spring Harbor Laboratory Press, Cold Spring Harbor, N.Y., 1988). Monoclonal antibodies can be isolated and purified from hybridoma cultures by techniques well known in the art.

In some embodiments, the antibody can be recombinantly produced, for example, by phage display or by combinatorial methods. Phage display and combinatorial methods can be used to isolate recombinant antibodies that bind to a polypeptide described herein, or a biologically active subunit or analog thereof (see, for example, Ladner et al., U.S. Pat. No. 5,223,409). Such methods can be used to generate human monoclonal antibodies.

Human monoclonal antibodies can also be generated using transgenic mice carrying the human immunoglobulin genes. Splenocytes from these transgenic mice immunized with the antigen of interest are used to produce hybridomas that secrete human mAbs with specific affinities for epitopes from a human protein (see, for example, Morrison et al., PNAS, 1984; 81:6851-6855).

A therapeutically useful antibody may be derived from a “humanized” monoclonal antibody. Humanized monoclonal antibodies are produced by transferring one or more CDRs from the heavy and light variable chains of a mouse (or other species) immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found, for example, in Jones et al. (Nature, 1986; 321:522) and Singer et al. (J. Immunol., 1993; 150:2844).

In addition, chimeric antibodies can be obtained by splicing the genes from a mouse antibody molecule with appropriate antigen specificity together with genes from a human antibody molecule of appropriate biological specificity; see, for example, Takeda et al. (Nature, 1985; 314:544-546). A chimeric antibody is one in which different portions are derived from different animal species.

Antibody fragments can be generated by techniques well known in the art. Such fragments include Fab fragments produced by proteolytic digestion, and Fab fragments generated by reducing disulfide bridges.

Antibodies, or fragments thereof, may be coupled directly or indirectly to a detectable marker by techniques well known in the art. A detectable marker is an agent detectable by spectroscopic, photochemical, biochemical, immunochemical, or chemical means. For example, useful detectable markers include fluorescent dyes, chemiluminescent compounds, radioisotopes, electron-dense reagents, enzymes, colored particles, biotin, or dioxigenin. A detectable marker often generates a measurable signal, such as radioactivity, fluorescent light, color, or enzyme activity.

When used for immunotherapy, antibodies may be unlabelled or labeled with a therapeutic agent. These agents can be coupled directly or indirectly to the monoclonal antibody by techniques well known in the art, and include such agents as drugs, radioisotopes, lectins, and toxins.

Antibodies useful in the present invention may be neutralizing antibodies. A neutralizing antibody is an antibody that decreases or completely removes the activity of a biologically active polypeptide described herein, or subunit or analog thereof. Neutralizing antibodies may, for instance, specifically bind to a region of a polypeptide that is part of the active site, and prevent the polypeptide and substrate to interact. The ability of an antibody to neutralize a polypeptide described herein can be evaluated by exposing a polypeptide having biological activity to an antibody and then measuring the activity of the polypeptide as described herein.

The present invention also provides compositions. A composition of the present invention may include at least one polypeptide described herein, or a subunit or analog thereof, or may include an antibody described herein. The compositions of the present invention may optionally further include a pharmaceutically acceptable carrier. “Pharmaceutically acceptable” refers to a diluent, carrier, excipient, salt, etc., that is compatible with the other ingredients of the composition, and not deleterious to the recipient thereof. Typically, the composition includes a pharmaceutically acceptable carrier when the composition is administered to a subject. The compositions of the present invention may be formulated in pharmaceutical preparations in a variety of forms adapted to the chosen route of administration, including routes suitable for stimulating an immune response to an antigen. Thus, a composition of the present invention can be administered via known routes including, for example, oral; parental including intradermal, transcutaneous and subcutaneous; intramuscular, intravenous, intraperitoneal, etc. and topically, such as, intranasal, intrapulmonary, intramammary, intravaginal, intrauterine, intradermal, and transcutaneous.

Solutions or suspensions can include the following components: a sterile diluent such as water for administration, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions (where water soluble) or dispersions and sterile powders for the extemporaneous preparation of sterile solutions or dispersions. For intravenous administration, suitable carriers include physiological saline, bacteriostatic water, Cremophor EL™ (BASF, Parsippany, N.J.) or phosphate buffered saline (PBS). A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate, and gelatin.

Sterile solutions can be prepared by incorporating the active compound (i.e., the polypeptide or antibody) in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Pharmaceutically compatible binding agents, and/or adjuvant materials can be included as part of the composition.

For administration by inhalation, the active compounds may be delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from, for instance, Alza Corporation and Nova Pharmaceuticals, Inc. Liposomal suspensions can also be used as pharmaceutically acceptable carriers. These can be prepared according to methods known to those skilled in the art.

In some aspects, a composition of the present invention may be administered in an amount sufficient to treat certain conditions as described herein. The amount of polypeptides present in a composition of the present invention can vary. For instance, the dosage of polypeptide can be between 0.01 micrograms (μg) and 300 mg, typically between 0.1 mg and 10 mg. For an injectable composition (e.g. subcutaneous, intramuscular, etc.) the polypeptide may be present in the composition in an amount such that the total volume of the composition administered is 0.5 ml to 5.0 ml, typically 1.0-2.0 ml. The amount administered will vary depending on various factors including, but not limited to, the specific polypeptides chosen, the weight, physical condition and age of the subject, and the route of administration. Thus, the absolute weight of the polypeptide included in a given unit dosage form can vary widely, and depends upon factors such as the species, age, weight and physical condition of the subject, as well as the method of administration. Such factors can be determined by one of skill in the art. For instance, in vitro and animal models are available that are used routinely and commonly accepted as models for the study of human disease. With respect to the study of human disease caused by R. prowazekii, a mouse model infected with the closely related R. typhi is a well characterized and commonly accepted animal model (see, for instance, Walker et al., Lab. Invest., 2000; 80:1361-72). The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans.

The compositions can be administered one or more times per day to one or more times per week, including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with an effective amount of a polypeptide can include a single treatment or, preferably, can include a series of treatments.

A composition including a pharmaceutically acceptable carrier can also include an adjuvant. An “adjuvant” refers to an agent that can act in a nonspecific manner to enhance an immune response to a particular antigen, thus potentially reducing the quantity of antigen necessary in any given immunizing composition, and/or the frequency of injection necessary in order to generate an adequate immune response to the antigen of interest. Adjuvants may include, for example, IL-1, IL-2, emulsifiers, muramyl dipeptides, dimethyl dioctadecyl ammonium bromide (DDA), pyridine, aluminum hydroxide, oils, saponins, alpha-tocopherol, polysaccharides, emulsified paraffins (including, for instance, those available from under the tradename EMULSIGEN from MVP Laboratories, Ralston, NB), ISA-70, RIBI and other substances known in the art. It is expected that polypeptides of the present invention will have immunoregulatory activity and that such polypeptides may be used as adjuvants that directly act as T and/or B cell activators or act on specific cell types that enhance the synthesis of various cytokines or activate intracellular signaling pathways.

In another embodiment, a composition of the invention including a pharmaceutically acceptable carrier can include a biological response modifier, such as, for example, IL-2, IL-4 and/or IL-6, TNF, IFN-alpha, IFN-gamma, and other cytokines that effect immune cells. An composition can also include other components known in the all such as an antibiotic, a preservative, an anti-oxidant, or a chelating agent.

The present invention provides methods for using the compounds described herein. In one aspect, the present invention is directed to methods for treating one or more symptoms caused by infection by an intracellular pathogen, preferably by a gram negative intracellular pathogen, more preferably, by a member of the genus Rickettsia, most preferably, by R. prowazekii. Preferably, the intracellular pathogen contains a homolog of the tlyC and/or pld described herein, such as R. typhi, R. rickettsii, R. conorii, R. akari, R. sibirica, Coxiella burnetii, Yersinia pestis, Yersinia pseudotuberculosis, Yersinia enterocolitica, Pseudomonas putida, Burkholderia cepacia, Salmonella typhimurium, Salmonella enterica, Proteus vulgaris, Klebsiella pneumoniae, Burkholderia mallei, Pseudomonas aeruginosa, Chlamydophila pneumoniae, Chlamydia trachomatis, Vibrio fischeri, Vibrio fischeri, Vibrio vulnificus, Vibrio cholerae, Bacteroides fragilis, Helicobacter pylori, Plasmodium chabaudi, Escherichia coli, Shigella flexneri, Plasmodium yoelii, Plasmodium falciparum, Francisella tularensis, Bordetella pertussis, Streptococcus pneumoniae, Streptococcus mutans, Streptococcus agalactiae, Streptococcus pyogenes, Ehrlichia ruminantium, Ehrlichia canis, Anaplasma marginale, Borrelia garinii, Borrelia burgdorferi, Clostridium perfringens, Bartonella hensalae, Bartonella quintana, Brucella melitensis, Brucella suis, Bacillus cereus, Bacillus anthracis, Bacteroides fagilis, Listeria monocytogenes, Haemophilus influenzae, Treponema pallidum, Campylobacter jejuni, Haemophilis ducrei, Neisseria meningitidis, Staphylococcus aureus, Neisseria gonorhoeae, Staphylococcus epidermidis, Legionella pneumophila, Enterococcus faecium, Mycobacterium tuberculosis, Chlamydia trachomatis, Clostridium tetani, Mycobacterium bovis, Bacillus cereus, Enterococcus faecalis, Leptospira interogans, Mycobacterium leprae, Corynebacterium diphtheriae, Plasmodium berghei, Fowlpox virus, Entamoeba histolytica, and Proteus mirabilis.

As used herein, the term “infection” refers to the presence of and multiplication of an intracellular pathogen in the body of a subject. The infection can be clinically unapparent, or result in symptoms associated with disease caused by the intracellular pathogen. The infection can be at an early stage, or at a late stage. The subject is a mammal, for instance, a human, a mouse, a rat, a guinea pig, or a non-human primate, preferably, a human. The method can include passive immunization, e.g., administering an effective amount of a composition including an antibody described herein, preferably a neutralizing antibody, to a subject having or at risk of having an infection, or symptoms of an infection, and determining whether at least one symptom of the infection is changed, preferably, reduced.

Treatment of symptoms associated with infection by an intracellular pathogen can be prophylactic or, alternatively, can be initiated after the development of an infection. As used herein, the term “symptom” refers to objective evidence in a subject of a condition caused by infection by an intracellular pathogen. Symptoms associated with infection by an intracellular microbe and the evaluation of such symptoms vary depending upon the microbe, and are routine and known in the art. Examples of symptoms of early stage infection by a Rickettsia include, for instance, fever, headache, muscle ache, nausea, loss of appetite, vomiting, or a combination thereof. Treatment that is prophylactic, for instance, initiated before a subject manifests symptoms of a condition caused by an intracellular pathogen, is referred to herein as treatment of a subject that is “at risk” of developing the condition. Typically, a subject “at risk” of developing a condition is a subject present in an area where others having the condition have been diagnosed and/or is likely to be exposed to an intracellular pathogen causing the condition. Accordingly, administration of a composition can be performed before, during, or after the occurrence of the infection. Treatment initiated after the development of an infection may result in decreasing the severity of the symptoms, or completely removing the symptoms. In this aspect of the invention, an “effective amount” is an amount effective to prevent the manifestation of symptoms of an infection, decrease the severity of the symptoms of an infection, and/or completely remove the infection.

In another aspect the present invention is directed to methods for inducing an immune response in a subject. The method includes administering to a subject an effective amount of a composition including a polypeptide described herein, or biologically active subunit or analog thereof, to a subject. The amount administered is effective to provide for an immune response that can protect the subject from infection by an intracellular pathogen, preferably by a gram negative intracellular pathogen, more preferably, by a member of the genus Rickettsia, most preferably, by R. prowazekii. As discussed above, the method can include more than one administration. Alternatively, the method can include administering to a subject an effective amount of a composition including a polynucleotide encoding a polypeptide described herein, or biologically active subunit or analog thereof, to a subject. Methods for making and using DNA vaccines are routine and well known (see, for instance, Barbet et al., U.S. Pat. No. 6,593,147).

Another aspect of the present invention is directed to methods for diagnosing in a subject an infection by an intracellular pathogen, preferably by a gram negative intracellular pathogen, more preferably, by a member of the genus Rickettsia, most preferably, by R. prowazekii. For instance, antibodies described herein can be used to identify the presence of a TlyC polypeptide or a Pld polypeptide, or a biologically active subunit or analog thereof, in a subject. The presence of such a polypeptide indicates the subject is infected with an intracellular pathogen. The method typically includes contacting a biological sample with an antibody that specifically binds a polypeptide described herein, or a subunit or analog thereof to form a polypeptide:antibody complex, and detecting the polypeptide:antibody complex.

A “biological sample” refers to a sample of tissue or fluid isolated from a subject, including but not limited to, for example, blood, plasma, serum, spinal fluid, lymph tissue and lymph fluid, samples of the skin, external secretions of the skin, respiratory, intestinal, and genitourinary tracts, tears, saliva, milk, blood cells, organs, and biopsies. A biological sample may be manipulated to enrich for a particular population of cells such as endothelial cells, macrophages, or leukocytes (see, for instance, La Scola et al., J. Clin. Microbiol., 1996; 34:2722-2727), and/or manipulated to lyse any cells present in the biological sample.

The assays for detecting infection by an intracellular pathogen, such as R. prowazekii, can be performed in any of several formats. For example, the presence of polypeptides that are specifically bound by the antibodies described herein can be detected using standard and well known electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include the use of antibody bound to a detectable marker for detecting the formation of a polypeptide:antibody complex.

In another aspect, a method for diagnosing in a subject an infection by an intracellular pathogen, preferably by a gram negative intracellular pathogen, more preferably, by a member of the genus Rickettsia, most preferably, by R. prowazekii, includes amplifying nucleotides of a coding region encoding a TlyC polypeptide or a Pld polypeptide, or amplifying mRNA encoded by such a coding region, to form amplified polynucleotides, and detecting the amplified polynucleotides. Preferably, nucleotides are amplified by a polymerase chain reaction (PCR) based method. In PCR, a molar excess of a primer pair is added to a biological sample that includes polynucleotides, preferably genomic DNA. The primers are extended to form complementary primer extension products which act as template for synthesizing the desired amplified polynucleotides. As used herein, the term “primer pair” means two oligonucleotides designed to flank a region of a polynucleotide to be amplified. The polynucleotide to be amplified can be referred to as the template polynucleotide. In some aspects, the template polynucleotide is the region encoding a TlyC polypeptide, preferably, the nucleotide sequence SEQ ID NO:1. In other aspects, the template polynucleotide is the region encoding a Pld polypeptide, preferably, the nucleotide sequence SEQ ID NO:3. In additional aspects, the target polynucleotide is an mRNA polynucleotide encoded by one of the coding regions. One primer is complementary to nucleotides present on one strand at one end of a template polynucleotide and another primer is complementary to nucleotides present on the other strand at the other end of the template polynucleotide. Those skilled in the art will recognize that primer pairs can be easily made using the sequence present at SEQ ID NO:1 or SEQ ID NO:3 and routine methods. A polynucleotide primer of this aspect of the invention includes, in increasing order of preference, at least 15 consecutive nucleotides, at least 18 consecutive nucleotides, at least 20 consecutive nucleotides, at least 24 consecutive nucleotides, or at least 27 consecutive nucleotides. Typically, a polynucleotide primer of this aspect of the invention has at least about 95% sequence identity, preferably at least about 97% sequence identity, most preferably, about 100% sequence identity with the target sequence to which the primer hybridizes.

After amplification, the sizes of the amplified polynucleotides may be determined, for instance by gel electrophoresis, and compared. The amplified polynucleotides can be visualized by staining (e.g., with ethidium bromide) or labeling with a suitable label known to those skilled in the art, including radioactive and nonradioactive labels. The conditions for amplifying a polynucleotide by PCR vary depending on the nucleotide sequence of primers used, and methods for determining such conditions are routine in the art.

The present application also provides methods for identifying agents that inhibit the activity of a polypeptide described herein including, for instance, high throughput assays. The method typically includes contacting a polypeptide and an agent to form a mixture, and determining whether the agent inhibits activity of the polypeptide. The sources for potential agents to be screened include, for instance, chemical compound libraries, fermentation media of bacteria and fungi, and cell extracts of plants and other vegetations. Without intending to be limiting, an agent can be, for instance, an organic compound, an inorganic compound, a metal, a polypeptide, a non-ribosomal polypeptide, a polyketide, or a peptidomimetic.

### Kits

The present invention also provides a kit for detecting infection by an intracellular pathogen, such as R. prowazekii. The kit includes antibodies discussed above in a suitable packaging material in an amount sufficient for at least one assay. Optionally, other reagents such as buffers and solutions needed to practice the invention are also included, such as a polypeptide described herein, or a subunit or analog thereof, for use as a positive control. As used herein, the phrase “packaging material” refers to one or more physical structures used to house the contents of the kit. The packaging material is constructed by well known methods, generally to provide a sterile, contaminant-free environment. The packaging material may have a label which indicates that the antibody can be used for identifying whether a subject in infected with an intracellular pathogen. In addition, the packaging material contains instructions indicating how the materials within the kit are employed. As used herein, the term package or container refers to a receptacle such as glass, plastic, paper, foil, and the like, capable of holding within fixed limits the antibody. Thus, for example, a package can be a plastic vial used to contain milligram quantities of an antibody. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

This example reports the establishment of a system for expression of rickettsial virulence factors in another intracellular pathogen, Salmonella enterica serovar Typhimurium. All Salmonella spp. possess the ability to invade host cells but lack the ability to escape phagosomal vacuoles, having adapted to replicating within a vacuole that has the characteristics of a late endosome (Catron et al., Cell. Microbiol., 2002; 4:315-328; Takeuchi, Am. J. Pathol., 1967; 50:109-136). We hypothesized that expression in Salmonella of the R. prowazekii gene responsible for phagosomal escape would complement the transformants with the ability to escape into the host cell cytosol.

In this study the period of active phagosomal escape was determined. It was demonstrated that both tlyC and pld, but not tlyA or pat1, were transcriptionally expressed in R. prowazekii during the peak time of escape. To further study the membranolytic functions of tlyC and pld, the genes were introduced into Salmonella isolates and the expression of each gene in the transformants was demonstrated by reverse transcriptase PCR (RT-PCR). A quantitative ultrastructural study demonstrated that Salmonella organisms expressing either tlyC or pld were able to escape phagosomal vacuoles, resulting in salmonellae exiting into the host cell cytosol.

Experimental design. In order to determine the period of active escape from the phagosome, Vero cells were infected with R. prowazekii and placed in Ito's fixative (a mixture of 1.25% formaldehyde, 2.5% glutaraldehyde, 0.03% trinitrophenol, 0.03% CaCl₂, and 0.05 M cacodylate buffer, pH 7.3 [Ito et al., Rickettsiae and Rickettsial Diseases, 1981, Academic Press, New York, N.Y., p. 213-227]) for electron microscopy or in RNAlater (Ambion, Austin, Tex.) for RT-PCR analysis at 30 and 50 minutes postinfection. The fixed cells were further studied by quantitative ultrastructural analysis. The proportion of rickettsiae within the host cell cytosol to the total number (at least 50 for each time point) of rickettsiae evaluated was determined. Rickettsiae in the process of escape were considered intracytosolic. The experiment was repeated in order to ensure reproducibility, and the results were calculated as the mean percentages A standard deviations for rickettsiae within the cytosol.

To determine which of the four membranolytic genes (pat1, tlyA, tlyC, or p/a) were expressed during the period of active rickettsial phagosomal escape, RT-PCR was performed using mRNA isolated from the cells fixed in RNAlater. The genes found to be transcribed during rickettsial escape were amplified from the genome by PCR, along with the putative promoter regions upstream of the open reading frame, and cloned into the pcr2.1-TOPO vector (Invitrogen, Carlsbad, Calif.). The plasmids were propagated in Top 10F′ E. coli (Invitrogen, Carlsbad, Calif.). E. coli clones that transcribed the rickettsial genes (tlyC or p/d) as determined by RT-PCR were chosen for plasmid purification using the High Pure plasmid isolation kit (Roche, Mannheim, Germany). The plasmids containing the genes were each electroporated into Salmonella enterica serovar Typhimurium (SB109) to determine whether or not the cloned genes could be involved in phagosomal escape. Salmonella was chosen for this experiment as it enters Vero cells but does not escape from phagosomal vacuoles, thereby allowing for the possibility of complementation of the ability to escape phagosomal vacuoles. RT-PCR was used again to confirm the transcriptional expression of the selected genes in Salmonella isolates. A quantitative ultrastructural analysis was performed exactly as described for the rickettsial study, except that observations were made at 4 hours after inoculation of cells. In both experiments, care was taken to avoid the counting of the same bacteria in serial sections.

Cells. Vero cells (African green monkey fibroblast cell line from the kidney) were obtained from the American Type Culture Collection (Manassas, Va.). Cells were cultivated in Dulbecco's minimum essential medium (Gibco, Grand Island, N.Y.), which contained 5% bovine calf serum (HyClone Inc., Logan, Utah) and 10 mM HEPES, in 150-cm²-surface-area flasks or 24-well plates (approximately 2×10⁵ cells per well) and were incubated at 37° C. in 5% CO₂. Vero cells were chosen for this study as they are nonphagocytic and therefore do not ingest dead organisms.

Rickettsia. Rickettsia prowazekii (Breinl strain), provided by G. A. Dasch, Naval Medical Research Institute (Bethesda, Md.), was passaged twice in yolk sacs of embryonated chicken eggs in our laboratory; 100 μl of infected yolk sac (10⁵ PFU per flask) was used to infect three 150-cm²-surface-area flasks of confluent Vero cells. The flasks were then incubated at 37° C. until they were 100% infected as determined by Protocol (Fisher, Pittsburgh, Pa.) staining of infected cells scraped from the monolayer. Stocks consisting of 1-ml aliquots of infected Vero cells were then prepared by scraping the infected cells from the monolayer and storing them in Dulbecco's minimum essential medium at −80° C. Five 1-ml aliquots were used to infect 10 150-cm²-surface-area flasks of confluent Vero cells, and the infection was monitored by Protocol staining of slides containing smears of infected cells from each flask. Once the infected cells were observed to contain more than 100 rickettsiae per cell, the monolayer was scraped from the flasks, and the rickettsiae were purified using Renografin density gradient centrifugation as previously reported (Hanson et al., Infect. Immun., 1981; 34:596-604). The light and heavy bands were combined into a 10-ml suspension of sucrose-phosphate-glutamic acid medium (0.128 M sucrose, 0.0038 M KH₂PO₄, 0.0072 M K₂HPO₄, 0.0049 M monosodium L-glutamic acid). The purified rickettsiae (4×10⁹ PFU/ml) were immediately used to infect 24-well plates containing confluent Vero cells for both determination of expression of selected genes by RT-PCR and quantitative electron microscopic studies. Vero cells were incubated at 37° C., harvested, and placed in either RNAlater for RT-PCR analyses or Ito's fixative for electron microscopy.

Salmonella. Salmonella enterica serovar Typhimurium strain SB 109, provided by Jorge Galan, Department of Cell Biology, Yale University (New Haven, Conn.). This Salmonella strain contains a mutation in the invE gene and has been demonstrated to be attenuated in the invasion of epithelial cells (Ginocchio et al., Proc. Natl. Acad. Sci. USA, 1992; 89:5976-5980). A qualitative ultrastructural study performed by our laboratory indicated that this Salmonella strain does invade Vero cells, with only a small portion of the organisms escaping from phagosomal vacuoles. Furthermore, it has been documented that several Salmonella species do efficiently invade Vero cells (Barrow et al., J. Med. Microbiol., 1989; 28:59-67). Salmonella was propagated in either imMedia Kan liquid (nontransformed Salmonella), imMedia Amp liquid (transformed Salmonella), or imMedia Amp plates (transformed Salmonella).

Primers for RT-PCR and PCR. Primers used in PCRs were designed to amplify regions within the potential membranolytic genes from DNA and RNA isolated from R. prowazekii: tlyA forward primer 5′-TGGGATTAAGGTGAAATAGACATGATCC-3′ (SEQ ID NO:5) (nucleotides 125 to 151); tlyA reverse primer 5′-GCCAATTCTTAATTTTATCACATACCT-3′ (SEQ ID NO:6) (nucleotides 616 to 643); pat1 forward primer 5′-CTTATTCTTATTTTACTTAATTTGCCG-3′ (SEQ ID NO:7) (nucleotides 169 to 195); pat1 reverse primer 5′-TATTCCTCCACCACAACAAACAGT-3′ (SEQ ID NO:8) (nucleotides 724 to 747); tlyC forward primer 5′-ATTGAAGCTGGAAGATAAAATTGTTGAAGATA-3′ (SEQ ID NO:9) (nucleotides 207 to 223); tlyC reverse primer 5′-TAGCTTTTCACCTATTATTTCTTCAAGC-3′ (SEQ ID NO:10) (nucleotides 693 to 720); pld forward primer 5′-ATGAAGAGCAAAAATAATAAATTTA-3′ (SEQ ID NO: 11) (nucleotides 1 to 25); and pld reverse primer 5′-CTAAAAATGTACTGCATTACTCGTTGTT-3′ (SEQ ID NO: 12) (nucleotides 591 to 618).

PCR amplification of entire open reading frames with the upstream endogenous promoters. A previous study by Policastro and Hackstadt indicated that the promoters for both of the rickettsial genes ompA and ompB function in E. coli, justifying the approach of using the endogenous rickettsial promoters (Policastro et al., Microbiology, 1994; 140:2941-2949). Primers used to amplify both tlyC and pld with the putative endogenous rickettsial promoters were designed based on the genomic nucleotide sequence of R. prowazekii (GenBank accession no. AJ235273): tlyC forward primer (nucleotides −163 to −135) 5′-ACTTTTGAGAATCATTTTATTCATATGT-3′ (SEQ ID NO:13) and reverse primer (nucleotides 888 to 912) 5′-CTATGTTAAATTATCACTATTCAA-3′ (SEQ ID NO:14) and pld forward primer (nucleotides −300 to −274) 5′-AGTAATGAGTGGTTTATGCAACGA-3′ (SEQ ID NO:15) and reverse primer (nucleotides 590 to 618) 5′-CTAAAAATGTACTGCATTACTCGTTGTT-3′ (SEQ ID NO: 16). For PCR, 0.60 μg of purified R. prowazekii (Breinl strain) DNA was used as the template. Thermal cycling conditions for all PCRs were as follows: 1 denaturation cycle at 95° C. for 5 minutes; 35 cycles of denaturation at 95° C. for 30 seconds, annealing at 52° C. for 30 seconds, and extension at 72° C. for 2 minutes; followed by 1 extension cycle at 72° C. for 5 minutes. Following PCR, 12 μl of the reaction mixture was electrophoretically separated on a 1% agarose gel and visualized by ethidium bromide staining. Accuracy of the DNA sequences was confirmed by sequencing using an ABI automated sequencer.

Cloning of tlyC and pld into E. coli and Salmonella. The PCR products, along with the promoters of both tlyC and pld, were cloned into the pcr2.1-TOPO vector (Invitrogen, Carlsbad, Calif.) and propagated in Top 10 F′ chemically competent E. coli according to the manufacturer's instructions (Invitrogen, Carlsbad, Calif.). Transformed E. coli organisms were plated onto imMedia Amp agar (Invitrogen, Carlsbad, Calif.). Positive clones were selected by blue/white screening.

Plasmids expressing pld or tlyC were isolated using the High Pure plasmid isolation kit (Roche, Mannheim, Germany) according to the manufacturer's instructions. Salmonella cells for transformation were grown overnight in imMedia Kan liquid (Invitrogen, Carlsbad, Calif.). Briefly, 1 ml of Salmonella organisms was mixed with at least 0.05 μg of pld plasmids, tlyC plasmids, or plasmids with no insert. Salmonellae were incubated with plasmids for 1 h and were then centrifuged for 1 min at 16,100×g in an Eppendorf 5415D centrifuge. The pellets were resuspended in 100 μl of a 10% glycerol solution and electroporated at 1.6 kV, 200Ω, and 25 μF for 4.0 milliseconds in 0.1-cm-gapped electroporation cuvettes (Eppendorf, Hamburg, Germany). The transformants were then grown on imMedia Amp agar plates overnight at 37° C. Salmonella clones were selected and grown overnight in imMedia Amp liquid (Invitrogen, Carlsbad, Calif.). RNA was isolated from 1-ml broth samples containing transformed Salmonella cells and tested for transcription of the rickettsial genes by RT-PCR.

RNA extraction and RT-PCR. Transformed E. coli colonies or transformed Salmonella colonies were selected and grown in imMedia Amp liquid overnight at 37° C. RNA isolation was performed by using an RNeasy kit according to the manufacturer's instructions (QIAGEN, Valencia, Calif.). RNA from R. prowazekii-infected Vero cells fixed in RNAlater was also extracted by the RNeasy kit. RNA extractions from all samples were separated electrophoretically on a 1% agarose gel, stained with ethidium bromide, and analyzed to ensure that the RNA was not degraded. All RT-PCRs were performed with the Titanium one-step RT-PCR kit (BD Biosciences, San Jose, Calif.). The PCR mixtures were incubated at 50° C. for 1 hour and 94° C. for 5 minutes, followed by 30 cycles of 95° C. (1 minute), 50° C. (30 seconds), and 72° C. (1 minute), and then 1 cycle at 72° C. (5 minutes). RT-PCRs were carried out with an RNA concentration of 10 ng/μl.

Infection assay. Salmonella (SB109 nontransformed) or the transformants were used to infect Vero cells in a 24-well plate at a multiplicity of infection of approximately 70:1. The infected cells were incubated for 4 hours at 37° C. in a 5% CO₂ atmosphere.

Electron microscopy. Vero cells infected with R. prowazekii for 30 or 50 minutes or with Salmonella for 4 hours were harvested and immersed in Ito's fixative overnight (Ito et al., Rickettsiae and Rickettsial Diseases, 1981, Academic Press, New York, N.Y., p. 213-227). Pellets were dehydrated in ethanol, embedded in epoxy resin (Poly/Bed 812; Polysciences, Inc., Warrington, Pa.), and polymerized at 60° C. overnight. Ultrathin sections (70 nm) were cut using a Reichert Ultracut S ultramicrotome, placed on copper grids, stained with uranyl acetate and lead citrate, and examined in a Phillips CM 100 electron microscope at 60 kV.

Quantitative electron microscopy. Rickettsia or Salmonella organisms were identified as being either completely within vacuoles or completely within the cytosol of the host cell. Bacteria within ruptured phagosomal vacuoles (identified as being in the process of escape) were counted as intracytosolic. Electron photomicrographs of infected Vero cells were taken at low magnifications between ×2,950 and ×5,200, and after prints were made, the ultrastructural locations of intracellular bacteria were determined. The experiment was performed three times, and the material from each experiment was examined to ensure reproducibility. At least 50 bacteria were examined at each time point in each experiment. The data were calculated as the mean percentages of intravacuolar and cytosolic bacteria, and standard deviations were calculated for each time point using Microsoft Excel. The proportion of intracytosolic rickettsiae at 30 minutes postinfection was statistically compared to that at 50 minutes postinfection (χ² test) with the program epiInfo 2002. The proportions of Salmonella organisms expressing either tlyC or pld that were within the host cell cytosol were statistically compared to numbers of Salmonella strain SB 109 organisms by the χ² test with the program epiInfo 2002. Values from both experiments were considered significantly different when P was <0.05.

### Results

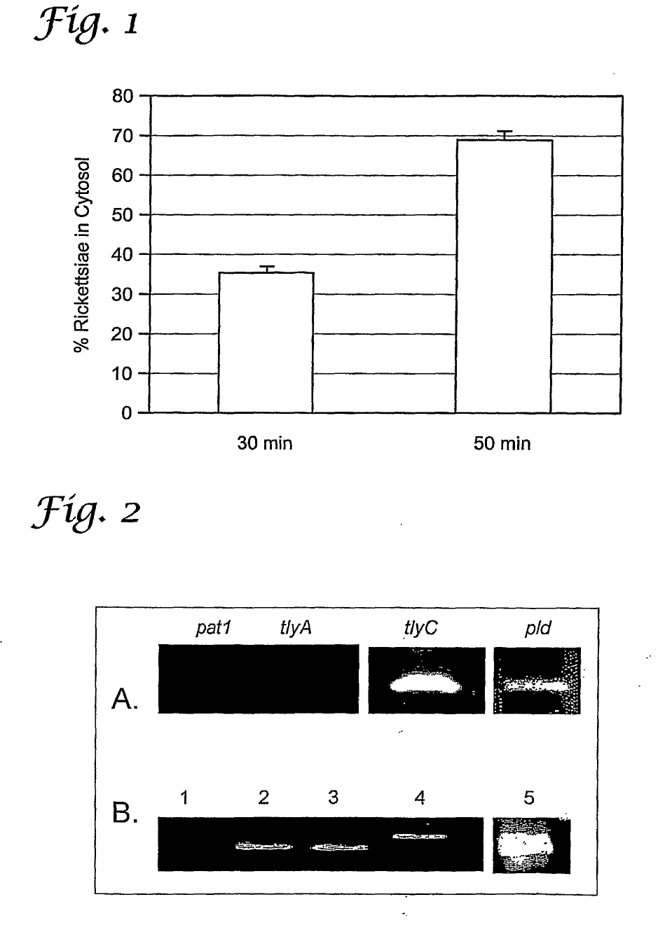

Period of phagosomal escape. The quantitative ultrastructural examination of Vero cells infected with R. prowazekii demonstrated that at 30 min postinfection, 35%±2.8% of rickettsiae had escaped from vacuoles, whereas at 50 min postinfection, 69%±2.1% of rickettsiae had escaped into the cytosol (FIG. 1). The differences between the percentages were statistically significant (P<0.001). These data demonstrated that between 30 and 50 min postinfection was a period of active phagosomal escape by R. prowazekii.

Evaluation of the transcription of potentially membranolytic genes and cloning of transcriptionally active genes into Salmonella. RT-PCR of RNA extracted from R. prowazekii-infected Vero cells with primers specific for pat1, tlyA, tlyC, and pld at 30 min postinfection revealed that only tlyC and pld were transcribed at 30 min postinfection and that tlyA and pat1 were not (FIG. 2). These results demonstrated that tlyC and pld, but not tlyA or pat1, were transcriptionally expressed during the period that most rickettsiae escape from phagosomal vacuoles into the host cell cytosol.

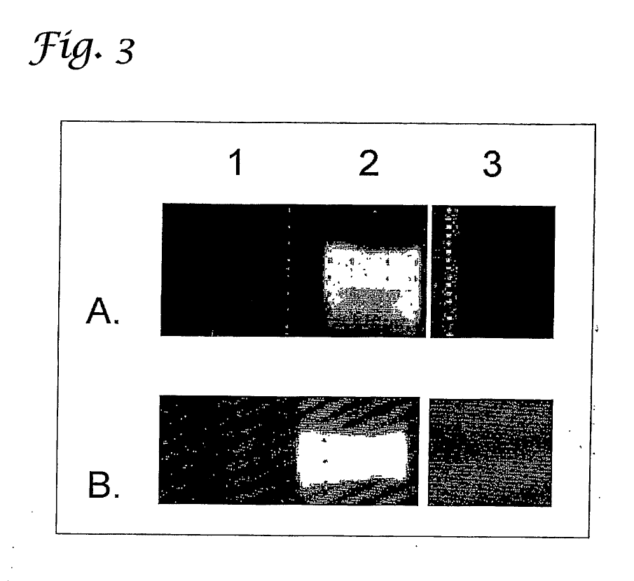

Primers specific for both tlyC and pld, including the entire gene and the upstream putative promoter regions, were used to amplify both genes by PCR from the genome of R. prowazekii. The PCR products were then ligated into the pcr2.1 vector. The vectors containing each gene were then separately electroporated into S. enterica serovar Typhimurium, and transcription was confirmed by RT-PCR (FIG. 3). The presence of mRNA of each gene demonstrated that both genes were actively transcribed in the transformed Salmonella.

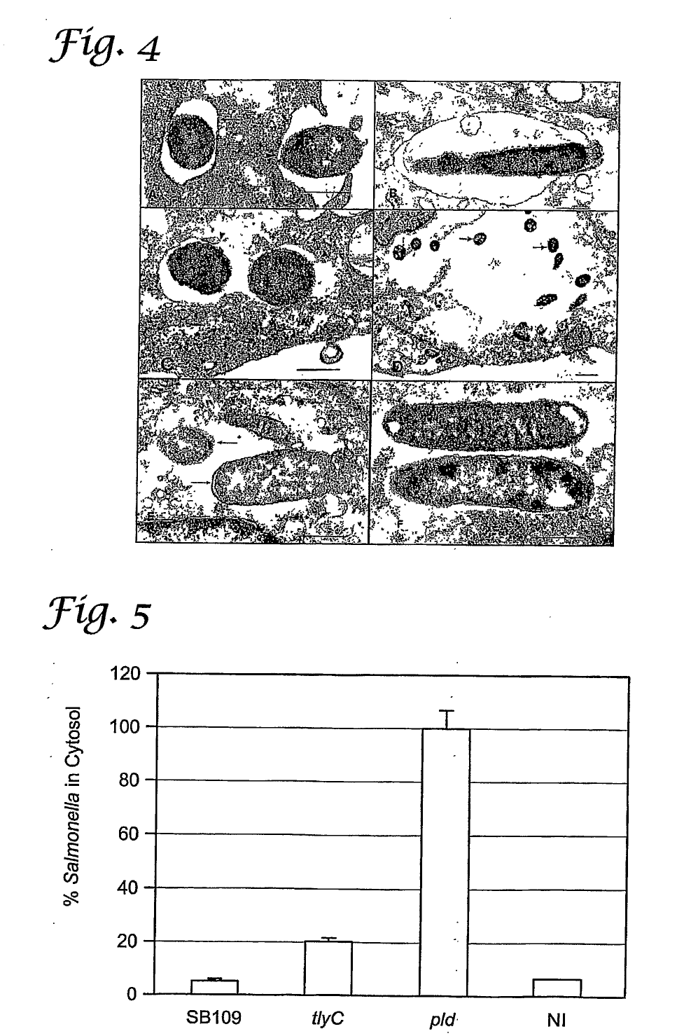

Infection of Vero cells and ultrastructural analyses. Vero cells were chosen for this study, as they are nonphagocytic and are therefore less likely to ingest dead organisms, an event that could skew the quantitative data because these bacteria would remain within host cell vacuoles. Vero cells infected with Salmonella transformants expressing either tlyC or pld were studied by electron microscopy at 4 hours postinfection (FIG. 4). Qualitative analyses revealed that while a portion of the tlyC-Salmonella transformants were observed free in the host cell cytosol, all pld-Salmonella transformants had relocated from phagosomal vacuoles to the cytosolic compartment. Nontransformed salmonellae and salmonellae with the nonrecombinant plasmid only were still predominantly located within vacuoles at 4 h postinfection. The quantitative ultrastructural analyses demonstrated that while approximately 20%±2.0% of the Salmonella organisms expressing tlyC escaped phagosomal vacuoles, 100% of Salmonella organisms expressing pld were present within the cytosol (FIG. 5). These findings were significantly different from each other (P<0.001). When percentages of tlyC-expressing or pld-expressing Salmonella transformants in the cytosol were statistically compared to the percentage of nontransformed Salmonella SB109 organisms in the cytosol, they were determined to be significantly different (P<0.001 and P<0.0000001, respectively); 5%±2.1% of nontransformed salmonellae and 6%±2.8% of salmonellae with vector alone were present in the cytosol. The difference between the controls was not statistically significant (P=0.08). These results demonstrated that Salmonella clones transcriptionally expressing either pld or tlyC escaped from phagosomal vacuoles and resided within the host cell cytosol.

### Discussion

The life cycle of Rickettsia spp. has been extensively studied by electron microscopy since the mid-1960s, resulting in the identification of several steps crucial to their intracellular survival and replication (Anacker et al., J. Bacteriol., 1967; 94:260-262; Anderson et al., J. Bacteriol., 1965; 90:1387-1404; Teysseire et al., Infect Immun., 1995; 63:366-374; Walker et al., Am. J. Trop. Med. Hyg., 2001; 65:936-942). There are two main groups of rickettsiae, the typhus group (R. prowazekii and R. typhi), and the spotted fever group (e.g., R. conorii and R. rickettsii). Rickettsiae from both groups adhere to host cells and induce phagocytosis by unknown mechanisms (Walker et al., Infect. Immun., 1978; 22:200-208). Rickettsial entry into host cells occurs by induced phagocytosis in nonphagocytic cells and by phagocytosis by phagocytic cells. Two outer membrane proteins have been hypothesized to function in adhesion, outer membrane protein A (OmpA; present only in the spotted fever group rickettsiae) and outer membrane protein B (OmpB; present in both the typhus and spotted fever groups) (Li et al., Microb. Pathog., 1998; 24:289-298; Uchiyama, Ann. N.Y. Acad. Sci., 2003; 990:585-590). Rickettsia may also adhere and enter by antibody-mediated opsonization and phagocytosis dependent on the presence of the Fc receptor on macrophages and endothelium and the Fc region of the antibody (Feng et al., Infect. Immun., 2004; 72:2222-2228; Feng et al., Infect. Immun., 2004; 72:3524-3530). Once within the host cell phagosomal vacuole, the microcapsular layer adjacent to the cell walls of nonopsonized rickettsiae appears to interact with the host cell phagosomal membrane. During this interaction, the vacuolar membrane becomes thicker and more osmiophilic. Eventually, large gaps appear in the host phagosomal membrane around the rickettsiae. Subsequent to this step, rickettsiae can be identified exiting the phagosomal vacuole into the host cell cytosol (Feng et al., Infect. Immun., 2004; 72:2222-2228; Feng et al., Infect. Immun., 2004; 72:3524-3530; Teysseire et al., Infect. Immun., 1995; 63:366-374; Walker et al., Am. J. Trop. Med. Hyg., 2001; 65:936-942). Once free in the cytosol, rickettsiae begin to multiply by binary fission. Spotted fever group rickettsiae polymerize actin by Arp2/3 nucleation that is dependent on the rickettsial protein RickA and use this ability to move intracellularly and to exit the cell via membrane-bound protrusions, both into adjacent cells and extracellularly (Heinzen et al., Infect. Immun., 1993; 61:1926-1935). It has been observed that spotted fever rickettsiae lyse the tips of the cell protrusions (Walker et al., Lab. Investig., 1980; 43: 388-396). Among typhus group rickettsiae, only R. typhi polymerizes short actin tails that do not create filopodia (Teysseire et al., Res. Microbiol., 1992; 143:821-829). Once typhus group rickettsiae multiply to high concentrations, they lyse the host cell membrane, also by an unknown mechanism.

Recent work in our laboratory has demonstrated that monoclonal antibodies, directed against OmpA or OmpB, or polyclonal antibodies are protective in the R. conorii mouse model, and cell culture experiments have demonstrated that the likely mechanism of protection is by inhibiting phagosomal escape (Feng et al., Infect. Immun., 2004; 72:2222-2228; Feng et al., Infect. Immun., 2004; 72:3524-3530). Ultrastructural and functional studies documented that rickettsiae unable to escape from the phagosomal vacuole undergo destruction by reactive oxygen species and reactive nitrogen species and by limitation of available tryptophan (Feng et al., Infect. Immun., 2000; 68:6729-6736).

Phagosomal escape is a crucial mechanism of intracellular survival for many bacterial pathogens, including Shigella spp. and Listeria spp. (Goebel et al., Curr. Opin. Microbiol., 2000; 3:49-53). Shigella spp. utilize the proteins IpaB, IpaC, and IpaD to mediate phagosomal escape and to disseminate to other cells (Page et al., Cell. Microbiol., 1999; 1:183-193; Picking et al., Infect. Immun., 2005; 73:1432-1440). Phagosomal escape by Listeria spp. has been the most widely studied of any set of bacteria. Listeriolysin O, encoded by the hlyA gene, is one mechanism of phagosomal escape, as demonstrated by complementation of this activity in Bacillus subtilis (Bielecki et al., Nature, 1990; 345:175-176). HlyA is hypothesized to function by causing oligomers to form pores within phagosomal vacuolar membranes, resulting in their rupture (Alouf, Int. J. Med. Microbiol., 2000; 290:351-356; Lety et al., Mol. Microbiol., 2002; 46:367-379). Other studies have suggested that two different phospholipase C enzymes may also be involved (Goebel et al., Curr. Opin. Microbiol., 2000; 3:49-53).

The mechanism of rickettsial phagosomal escape was originally hypothesized to be phospholipase A dependent (Walker et al., Am. J. Trop. Med. Hyg., 2001; 65:936-942; Winkler et al., Infect. Immun., 1982; 38:109-113). But to date, no phospholipase A gene has been identified in any of the sequenced rickettsial genomes (Andersson et al., Nature, 1998; 396:133-140; McLeod et al., J. Bacteriol., 2004; 186:5842-5855; Ogata et al., Science, 2001; 293:2093-2098). The most direct technique to determine rickettsial gene function would be the creation of clones of rickettsiae with the genes of interest inactivated. However, members of the genus Rickettsia have been very resistant to site-directed genetic manipulation (Baldridge et al., Appl. Environ. Microbiol., 2005; 71:2095-2105; Qin et al., Appl. Environ. Microbiol., 2004; 70:2816-2822; Rachek et al., J. Bacteriol., 1998; 180:2118-2124; Troyer et al., Infect. Immun., 1999; 67:3308-3311). This obstacle has required researchers to employ novel approaches to study Rickettsial gene function, namely, the expression of rickettsial genes in other bacteria (Radulovic et al., Infect. Immun., 1999; 67:6104-6108). Bacteria that lack specific rickettsial functions are chosen with the aim of complementing the function by introducing the appropriate rickettsial gene. In keeping with this strategy, we report here the transformation and expression of two R. prowazekii genes in Salmonella enterica serovar Typhimurium (SB 109 strain). Only two of the four potentially membranolytic genes, tlyC and pld, were expressed in R. prowazekii during the period of rickettsial escape from the phagosome. TlyC was first characterized by Radulovic et al., who demonstrated that the transformation of a nonhemolytic bacterium (Proteus mirabilis) with tlyC resulted in a hemolytic phenotype (Radulovic et al., Infect. Immun., 1999; 67:6104-6108). Renesto et al. expressed Pld in vitro and demonstrated it to possess phospholipase D activity by the catalysis of phosphatidyl choline, leading to the byproduct choline (Renesto et al., J. Infect. Dis., 2003; 188:1276-1283). Both TlyC and Pld are secreted and have been hypothesized to play a role in phagosomal escape by rickettsiae (Radulovic et al., Infect. Immun., 1999; 67:6104-6108; Renesto et al., J. Infect. Dis., 2003; 188:1276-1283). We have extended the knowledge of these genes by transcriptionally expressing them in Salmonella, resulting in transformants that escape phagosomal vacuoles by 4 h postinfection. TlyC probably plays a less important role in phagosomal escape, as suggested by our studies, and Pld is likely to be the major effector of rickettsial phagosomal escape (FIG. 5). The possibility that two rickettsial genes can carry out the crucial function of phagosomal escape suggests that redundancy and possibly synergy may exist in the performance of this specific function.

The presence of orthologs of tlyC in R. typhi (RT0725), R. conorii (RC1141), and Rickettsia rickettsii (Rick02001360) and of pld orthologs in R. typhi (RT0807), R. conorii (RC1270), and R. rickettsii (Rick02001502) indicates that antibodies or drugs targeting these proteins could be protective against other rickettsial species.

These findings offer the opportunity to identify compounds that interrupt the functions of TlyC or Pld. We believe that the development of drugs or antibodies that inhibit the function of TlyC or Pld could provide a novel therapeutic approach to treat infection with R. prowazekii which may have been engineered or selected to be resistant to tetracyline and chloramphenicol, as would potentially occur in a bioterrorist attack (Weiss et al., J. Bacteriol., 1962; 83:409-414).

### Example 2

### Inhibition of Phagosomal Escape in Cell Culture

Rickettsia prowazekii is grown in cell culture until there are approximately 200 rickettsiae per cell. The rickettsia are then renograffin purified and split into four groups. Group one is incubated with polyclonal antibodies against the Phospholipase-D (Pld) enzyme at 37° C. for three hours. Group two is incubated with antibodies against the TlyC protein at 37° C. for three hours. Group three is incubated with buffer at 37° C. for three hours as a negative control to demonstrate normal kinetics of escape. Group four is incubated with polyclonal antibodies against OmpB (outer membrane protein B) as a positive control since this antibody has been demonstrated to inhibit phagosomal escape. Each group is cooled to 4° C. and placed into wells of a 24-well tray of confluent Vero cells held at 4° C. The tray is centrifuged at 300 g for ten minutes to allow the rickettsiae to be synchronized and attached to the cell surface of the host cells. The 4° C. time point serves as time point zero minutes as they attach but not invade the cells. Media incubated at 37° C. is then placed in each well, allowing the rickettsia to invade. At 0, 10 minutes, 30 minutes, 1 hour, and 4 hours post-infection the cells are fixed in standard electron microscopy fixative. Cells are then processed for electron microscopy and observed at each time point. Rickettsiae within each sample are counted as being within a vacuole bound compartment or present within the cytosol and the groups are statistically compared. It is expected that the antibodies present within the phagosomes inhibit the functions of the proteins (TlyC and/or Pld) and result in a significant decrease of the number of rickettsiae present within the cytosol when compared to the control group.

### Example 3

### Passive Immunization of Mice with Polyclonal Antibodies Against Pld and TlyC

Due to the lack of a “true” animal model for R. prowazekii, this experiment will be performed in C3H/Hen mice using R. typhi. The genomes of R. typhi and R. prowazekii are nearly identical, and like R. prowazekii, R. typhi is a member of the spotted fever group. Polyclonal antibodies against R. prowazekii Pld and TlyC is expected to cross react with the R. typhi homologues as they share 95% amino acid homology. Mice are inoculated with 10 LD₅₀ of R. typhi, or buffer alone. On day 5 post infection the mice are given antibodies against OmpB as a positive control since this has been demonstrated to play a protective role in the mouse model, antibodies against Pld, antibodies against TlyC, or non-immune serum as a negative control (should result in lethality). The mice are observed in order to determine length of time until death. It is expected that the antibodies against Pld and TlyC results in protection of the mice and/or increase survival times. Mice are sacrificed at 24 and 48 hours post-antibody inoculation in order to compare numbers of rickettsia in lungs, liver, and spleen.

### Example 4

### Diagnosis of Rickettsial Infection in Mice

Mice from the previous experiment are sacrificed on days 0, 1, 2, 3, 4, and 5 post-infection. Blood is collected, and lysed by treating with NuPAGE LDS Sample Buffer (4×) at 65° C. for ten minutes. Crude lysate is resolved on an SDS-PAGE polyacrylamide gel at 170V for 1 hour. Controls of TlyC protein and Pld protein are included in different lanes. The gel is transferred to a nitrocellulose membrane and blocked over night with 3% milk/buffer. The membrane is then be probed with TlyC and Pld antibodies, and then incubated with alkaline phosphatase tagged rabbit anti mouse antibody and developed using standard methods. It is expected that the secreted proteins is detected in the blood of the infected animals. Blood from these mice is also screened with primers specific for the pld and tlyC genes in order to determine if the presence of the genes can be detected by polymerase chain reaction and/or real time polymerase chain reaction.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising:
providing a biological sample obtained from a subject suspected of having a Rickettsia infection;
contacting the biological sample with an antibody to form an antibody-polypeptide complex, wherein the antibody specifically binds a polypeptide having at least 95% identity to an amino acid sequence SEQ ID NO:2; and
detecting the antibody-polypeptide complex, wherein the presence of the antibody-polypeptide complex indicates the presence of a member of the genus Rickettsia in the subject.

2. The method of claim 1 wherein the biological sample is a blood sample.

3. The method of claim 1 wherein the antibody is a polyclonal antibody.

4. The method of claim 1 wherein the Rickettsia is R. prowazekii.

5. The method of claim 1 wherein the antibody does not specifically bind a polypeptide having less than 95% identity to the amino acid sequence SEQ ID NO:2.

6. A method comprising:
providing a biological sample obtained from a subject suspected of having a Rickettsia infection;
contacting the biological sample with an antibody to form an antibody-polypeptide complex, wherein the antibody specifically binds a polypeptide having at least 95% identity to an amino acid sequence SEQ ID NO:4; and
detecting the antibody-polypeptide complex, wherein the presence of the antibody-polypeptide complex indicates the presence of a member of the genus Rickettsia in the subject.

7. The method of claim 6 wherein the biological sample is a blood sample.

8. The method of claim 6 wherein the antibody is a polyclonal antibody.

9. The method of claim 6 wherein the Rickettsia is R. prowazekii.

10. The method of claim 6 wherein the antibody does not specifically bind a polypeptide having less than 95% identity to the amino acid sequence SEQ ID NO:4.

11. A method comprising:
providing a biological sample obtained from a subject suspected of infection with an intracellular pathogen;
contacting the biological sample with an antibody to form an antibody-polypeptide complex, wherein the antibody specifically binds a polypeptide having at least 80% identity to an amino acid sequence SEQ ID NO:2; and
detecting the antibody-polypeptide complex, wherein the presence of the antibody-polypeptide complex indicates the presence of an intracellular pathogen in the subject.

12. The method of claim 11 wherein the biological sample is a blood sample.

13. The method of claim 11 wherein the antibody is a polyclonal antibody.

14. The method of claim 11 wherein the intracellular pathogen is a gram negative intracellular pathogen.

15. A method comprising:
providing a biological sample obtained from a subject suspected of infection with an intracellular pathogen;
contacting the biological sample with an antibody to form an antibody-polypeptide complex, wherein the antibody specifically binds a polypeptide having at least 80% identity to an amino acid sequence SEQ ID NO:4; and
detecting the antibody-polypeptide complex, wherein the presence of the antibody-polypeptide complex indicates the presence of an intracellular pathogen in the subject.

16. The method of claim 15 wherein the biological sample is a blood sample.

17. The method of claim 15 wherein the antibody is a polyclonal antibody.

18. The method of claim 15 wherein the intracellular pathogen is a gram negative intracellular pathogen.

19. A method comprising:
administering an effective amount of a composition comprising an antibody to a subject, wherein the subject has or is at risk of having an infection by an intracellular pathogen, and wherein the antibody specifically binds a polypeptide having at least 80% identity to an amino acid sequence SEQ ID NO:2.

20. The method of claim 19 wherein the antibody specifically binds a polypeptide having at least 95% identity to an amino acid sequence SEQ ID NO:2.

21. The method of claim 19 wherein the intracellular pathogen is a gram negative intracellular pathogen.

22. The method of claim 21 wherein the gram negative intracellular pathogen is a member of the genus Rickettsia.

23. A method comprising:
administering an effective amount of a composition comprising an antibody to a subject, wherein the subject has or is at risk of having an infection by an intracellular pathogen, and wherein the antibody specifically binds a polypeptide having at least 80% identity to an amino acid sequence SEQ ID NO:4.

24. The method of claim 23 wherein the antibody specifically binds a polypeptide having at least 95% identity to an amino acid sequence SEQ ID NO:4.

25. The method of claim 23 wherein the intracellular pathogen is a gram negative intracellular pathogen.

26. The method of claim 25 wherein the gram negative intracellular pathogen is a member of the genus Rickettsia.

27. A method comprising:
administering to a subject an effective amount of a composition comprising a polypeptide, or a biologically active subunit or analog thereof, to a subject, wherein the polypeptide, or a biologically active subunit or analog thereof, has at least 80% identity to an amino acid sequence SEQ ID NO:2, and wherein the amount administered is effective to result in an immune response.

28. The method of claim 27 wherein the polypeptide, or a biologically active subunit or analog thereof, has at least 95% identity to an amino acid sequence SEQ ID NO:2

29. A method comprising:
administering to a subject an effective amount of a composition comprising a polypeptide, or a biologically active subunit or analog thereof, to a subject, wherein the polypeptide, or a biologically active subunit or analog thereof, has at least 80% identity to an amino acid sequence SEQ ID NO:4, and wherein the amount administered is effective to result in an immune response.

30. The method of claim 29 wherein the polypeptide, or a biologically active subunit or analog thereof, has at least 95% identity to an amino acid sequence SEQ ID NO:4.

31. An antibody that specifically binds a polypeptide having at least 95% identity to an amino acid sequence SEQ ID NO:2 and does not specifically bind a polypeptide having less than 95% identity to the amino acid sequence SEQ ID NO:2.

32. A composition comprising the antibody of claim 31.

33. An antibody that specifically binds a polypeptide having at least 95% identity to an amino acid sequence SEQ ID NO:4 and does not specifically bind a polypeptide having less than 95% identity to the amino acid sequence SEQ ID NO:4.

34. A composition comprising the antibody of claim 33.

35. A method of making an antibody, the method comprising:
administering to an animal an effective amount of polypeptide, or a biologically active subunit or analog thereof, to a subject, wherein the polypeptide, or a biologically active subunit or analog thereof, has at least 95% identity to an amino acid sequence SEQ ID NO:2, and wherein the amount administered is effective to result in an immune response; and
isolating antibody that specifically binds the polypeptide, or a biologically active subunit or analog thereof.

36. A method of making an antibody, the method comprising:
administering to an animal an effective amount of polypeptide, or a biologically active subunit or analog thereof, to a subject, wherein the polypeptide, or a biologically active subunit or analog thereof, has at least 95% identity to an amino acid sequence SEQ ID NO:4, and wherein the amount administered is effective to result in an immune response; and
isolating antibody that specifically binds the polypeptide, or a biologically active subunit or analog thereof.

37. A kit for detecting the presence of Rickettsia polypeptides in a biological sample, comprising an antibody, wherein the antibody specifically binds a polypeptide having at least 95% identity to an amino acid sequence SEQ ID NO:2.

38. A kit for detecting the presence of Rickettsia polypeptides in a biological sample, comprising an antibody, wherein the antibody specifically binds a polypeptide having at least 95% identity to an amino acid sequence SEQ ID NO:4.
